(19) United States
(12) Patent Application Publication
(10) Pub. No.: US 2022/0233627 A1
(43) Pub. Date: Jul. 28, 2022

(54) COMPOSITIONS COMPRISING PLANT EXTRACTS AND OILS AND RELATED METHODS OF TREATMENT AND THEIR PREPARATION

(71) Applicant: Le Nadur, LLC, Reading, MA (US)

(72) Inventors: Denise M. O'Hara, Reading, MA (US); Mengmeng Wang, Andover, MA (US)

(21) Appl. No.: 17/610,507

(22) PCT Filed: May 13, 2020
(86) PCT No.: PCT/US2020/032763

§ 371 Date: Nov. 11, 2021

Related U.S. Application Data
(60) Provisional application No. 62/847,786, filed on May 14, 2019.

(51) Int. Cl. A61K 36/899; A61K 36/185; A61K 36/23; A61K 36/28; A61K 36/42; A61K 36/53; A61K 36/57; A61K 36/738; A61K 36/74; A61K 36/886; A61K 47/06; A61K 47/46; A61K 31/355; A23L 33/105; A23L 33/115

(52) U.S. Cl. CPC A61K 36/899; A23L 33/115; A61K 36/23; A61K 36/28; A61K 36/42; A61K 36/53; A61K 36/57; A61K 36/738; A61K 36/74; A61K 36/886; A61K 47/06; A61K 47/46; A61K 31/355; A23L 33/105; A61K 36/185

(57) ABSTRACT

Disclosed are compositions comprising botanical extracts and cold-pressed oils and related methods of manufacture and methods of use for the treatment of skin conditions and diseases.

### RELATED APPLICATIONS PARAGRAPH

This application claims the benefit of U.S. Provisional Application No. 62/847,786, filed May 14, 2019. The entire teachings of the above application are incorporated herein by reference.

### BACKGROUND OF THE INVENTION

The skin is a complex organ with important functions for host physiology, with a protective physical barrier, the skin epidermis, that is dependent on junction adhesions molecules and tight junction proteins. Disruption of the expression or function of these components of the skin's physical barriers results in skin disorders and can contribute to inflammatory conditions in the skin. For example, the skin of patients with atopic dermatitis have reduced expression levels of zonula occludins-1 (ZO-1) and claudin-1, while knockdown of claudin-1 in mice induces a psoriasis-like condition.

In addition, skin-resident cells contribute to tissue homeostasis, defense and repair. During infection or tissue damage, resident immune cells in the skin and those infiltrating from the periphery interact and create an intricate defense network to resolve the challenge. T and B lymphocytes, non-immune cells (i.e., keratinocytes and fibroblasts) and Langerhans cells, a myeloid cell type, in the epidermis are involved in disorders such as psoriasis and atopic dermatitis, producing inflammatory cytokines during disease or irradiation. Excess production of pro-inflammatory cytokines are involved in skin diseases (for example, IL-1, IL1-b IL-6, IL8, IL17, IL18, IL33, alpha and beta interferon and tumor necrosis factor (TNF), MIP-1b, G-CSF), anti-inflammatory cytokines (for example, IL-4, IL-10, IL-13) and inflammation regulatory molecules prostaglandins (PGD, PGE and PGF), postacyclins (PGI), thromboxanes (TXA) all have a role.

The skin is highly innervated by sensory nerves expressing receptors that sense itch (pruriceptors). Transient receptor potential (TRP) A1 and TRPV1 function in pruritus of the skin in addition to histamine and cytokines, and are thought to be involved in pruritis.

Acute and chronic inflammatory diseases of the skin are widespread. Common symptoms may include mild to intense itchiness (pruritis), redness, swelling, heat and pain, sometimes with flaking and unsightly rashes, thickened scales (parakeratosis) and/or lesions.

Skin disorders have been shown to negatively affect a patient's quality of life, with patients suffering high social and economic costs and increased psychological burdens, morbidity and mortality, the latter due to inflammatory, cardiovascular, metabolic and neuropsychiatric comorbidities. Treatment options for skin inflammation and pruritis include topical creams and lotions, which function as an emollient to moisturize and soften the skin, and are typically paraffin based and do not address the underlying inflammation or pruritis. These creams and lotions often are not suitable for babies and cannot be used on broken skin. Other traditional approaches include, for example, steroid creams (e.g., clobetasol propionate), methotrexate and/or cyclosporine, that provide temporary relief, though are used for less than 2 weeks at a time and excessive use is not ideal due to the significant safety concerns for long term use such as thinning, blistering, burning, itching and peeling skin. The steroid betamethasone-17-valerate was shown to reduce acute dermatitis though did not alter patient-related symptoms of pain, itching, irritation, local heat and tightness. Alternative treatment options for chronic and severe skin disorders include antibody-based anti-inflammatory agents that are administered systemically rather than topically. However, treatment failure of these biopharmaceuticals may be attributable to loss of response over time, risk of infection, allergic, immunological or other unwanted reactions, in addition to drug compliance and drug side effects that complicate the treatment of most skin disorders. Most treatment options are immunosuppressive. In skin disorders where there are signs of infection antibiotics are commonly used, though the use of such antibiotics are frequently associated with disruption of the patients gut microbiome, which needs to be re-established following the treatment regimen. In severe skin disorders (e.g., Hidradenitis suppurativa (HS)) laser therapy or surgery may be needed to remove the affected skin.

Despite the prevalence of skin disorders there is no standard approach for its prevention or management that addresses the disruption and cellular balance between the dermis and epidermis. Inflammation and pruritis of the skin involve complex biological systems that include resident and trafficking cells, and a plethora of molecules ranging from chemotactic proteins, cytokines, inflammatory mediators and regulatory molecules. The complexity in biological systems of inflammation and pruritis may benefit from a combinatorial and combined approaches, instead of a single targeted biopharmaceutical approach, stimulating and balancing the adaptive and innate immune system of the patient depending on the skin situation.

### SUMMARY OF THE INVENTION

The present invention relates to compositions comprising botanical extracts (BE) and, optionally, cold-pressed oils for skin and hair-care preparations.

In some embodiments, the compositions disclosed herein comprise one or more of pomegranate fruit extract (Punica granatum), oats extract (Avena sativa), pennywort extract (Centella asiatica), sun flower seeds extract (Helianthus annuus), lemon balm extract (Melissa officinalis) and star anise extract (Illicium veraum) combined with aloe vera oil (Barbados aloe), rosehip oil (Rosa canina), sea buckthorn seed oil (Hippophae rhamnoides), pumpkin oil (Cucurbita pepo), pomegranate seed oil (Punica granatum), carrot seed oil (Daucus carota), chamomile oil (Matricaria chamomilla), coffee bean oil (Coffea arabica), raspberry seed oil (Rubus idaeus) and Vitamin E oil (gamma-tocopherol), and in certain embodiments are useful for personal care product preparations (e.g., cosmetic, dermatological). For example, in certain aspects, the compositions disclosed herein are useful for the inclusion in skin, scalp and hair treating preparations for cosmetic and/or dermatological uses, particularly for soothing irritations, inflammation and associated heat and pain from inflammation, itching, flaking of skin and scalp and overall treatment of tissue repair in inflammatory skin diseases and immune responses in tissue injuries.

The compositions disclosed herein can be prepared as skin and hair-care preparations; body and face creams and masks, lotions, toners; ointments; balms; gels, sprays; hair shampoos, hair lotions; foam baths, shower baths, bath soaking salts; powder and soaps.

Some aspects of the present invention are related to compositions comprising one or more botanical extracts and one or more cold-pressed oils, wherein: (a) the one or more botanical extracts are selected from the group consisting of pomegranate fruit (Punica granatum) extract, oats (Avena sativa) extract, pennywort (Centella asiatica) extract, sun flower seed (Helianthus annuus) extract, lemon balm (Melissa officinalis) extract, and star anise (Illicium verum) extract; and (b) the one or more cold-pressed oils are selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

In some embodiments, the one or more botanical extracts are aqueous botanical extracts, fermented botanical extracts, aqueous/alcoholic botanical extracts, alcoholic botanical extracts, organic solvent botanical extracts, enzymatic botanical extracts, acidic botanical extracts, pressure extracted botanical extracts, botanical extracts prepared by distillation, and/or botanical extracts prepared via saponification. In some embodiments, the one or more botanical extracts are a combination of aqueous botanical extracts and fermented botanical extracts.

In some embodiments, the compositions disclosed herein are in the form of an emulsion. In some embodiments, the compositions comprise at least two, at least three, at least four, at least five, or all six botanical extracts. In some embodiments, the compositions comprise a combination of all six botanical extracts as aqueous botanical extracts and as fermented botanical extracts. In some embodiments, the aqueous botanical extracts and fermented botanical extracts are combined at a ratio of between about 2:1 to 1:2, preferably at a ratio of 1:1. In some embodiments, the emulsion comprises at least two, at least three, at least four, at least five, at least six, at least seven, at least eight, or all nine cold-pressed oils.

In some embodiments, the fermented botanical extracts have been fermented with lactic acid bacteria. In some embodiments, the lactic acid bacteria are selected from the group consisting of Lactobacillus sakei, Lactobacillus brevis and combinations thereof.

In some embodiments, the compositions disclosed herein comprise about 8.8% by volume botanical extract (e.g., total amount of botanical extract) and about 12-14% by volume cold-pressed oil (e.g., total amount of cold-pressed oil). In some embodiments, the compositions further comprise pro-vitamin B5, tea tree (Melaleuca alternifolia) oil, jojoba (Simmondsia chinensis) oil, argan (Argania spinose) oil, and/or vitamin E oil.

In some embodiments, the compositions (e.g., topical compositions) are formulated as a paste, gel, cream, lotion, powder, soap, mask, or spray. In some embodiments, the compositions are formulated as a skin or hair care product. In some embodiments, the compositions are formulated for topical administration. In some embodiments, the composition are formulated as a lotion or cream.

Some aspects of the present invention are related to a methods of manufacturing the compositions disclosed herein, such methods comprising the following steps: (a) obtaining one or more aqueous botanical extracts by heating (e.g., at about 100° C. for about 45 minutes or more) an aqueous solution comprising botanical material from one or more plants selected from the group consisting of pomegranate fruit (punica granatum), oats (Avena sativa), pennywort (Centella asiatica), sun flower seed (Helianthus annuus), lemon balm (Melissa officinalis), and star anise (Illicium verum); (b) obtaining one or more fermented botanical extracts by fermenting an aqueous solution comprising botanical material from one or more plants selected from pomegranate fruit, oats, pennywort, sun flower seed, lemon balm, and star anise; (c) removing insoluble material from the one or more botanical extracts obtained in steps (a) and (b) and combining the resultant extracts, or combining the botanical extracts of (a) and (b) and removing insoluble material from the combined botanical extract; and (d) emulsifying the product of step (c) with one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil and Vitamin E oil (gamma-tocopherol).

In some embodiments of step (b), one or more fermented botanical extracts are obtained by fermenting an aqueous solution comprising botanical material from one or more plants selected from the group consisting of pomegranate fruit (punica granatum), oats (Avena sativa), pennywort (Centella asiatica), sun flower seed (Helianthus annuus), lemon balm (Melissa officinalis), and star anise (Illicium verum) with lactic acid bacteria for a suitable time (e.g., at about 20-24° C. for about 36-72 hours), followed by heat inactivation of the bacteria (e.g., at about 56° C. for about 30 minutes or more).

In some embodiments, step (c) further comprises emulsifying the aqueous botanical extract after removal of the insoluble material with one or more cosmetically acceptable solid oils, humectants, thickeners, and/or emulsifying agents. In some embodiments, the fermentation of step (b) is performed at about 20-24° C. for about 36 to 72 hours. In some embodiments, the fermentation is stopped by heat inactivation (e.g., at about 56° C. for about 30 minutes or more).

In some embodiments, the insoluble material in step (c) is removed by filtration or centrifugation. In some embodiments, provitamin B5 is added to the one or more aqueous botanical extracts prior to step (d). In some embodiments, the pro-vitamin B5 is provided in tea tree (Melaleuca alternifolia) oil, jojoba (Simmondsia chinensis) oil, argan (Argania spinose) oil, and/or vitamin E oil. In some embodiments, the one or more aqueous botanical extracts are cooled to about 40° C. or less prior to step (d).

Some aspects of the present invention are related to methods of manufacturing the compositions disclosed herein, such methods comprising the following steps: (a) providing one or more botanical extracts; (b) optionally removing insoluble material from the one or more botanical extracts obtained in step (a); and (c) emulsifying the product of step (b) with one or more cold-pressed oils selected from the group of cold-pressed oils consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil and Vitamin E oil (gamma-tocopherol).

In some embodiments, the one or more botanicals extracts of step (a) are prepared by aqueous extraction, fermentation, aqueous/alcoholic extraction, alcoholic extraction, organic solvent extraction, enzymatic extraction, acidic (e.g., vinegar) extraction, pressure extraction, extraction via distillation, and/or extraction via saponification.

Some aspects of the present disclosure are related to methods of treating a subject having a skin disease, skin disorder or skin condition, such methods comprising the topical administration of the compositions of the present invention. In some embodiments, the compositions are administered one or more times per day. In some embodiments, the composition is a cream or lotion. In some embodiments, the skin disease, disorder or condition is selected from the group consisting of hidradenitis suppurativa (HS), acne inversa, psoriasis, eczema, dry skin, sunburn, soft tissue injury, insect bite, radiation dermatitis, birth marks and moles, planter's wart, skin rash, acne vulgaris, dermal scaring and keloids from acne, dermal scaring and keloids from cutaneous burn injuries, skin peeling and cracking, and seborrheic dermatitis.

Some aspects of the present disclosure are related to compositions comprising extracts of pomegranate fruit (Punica granatum), oats (Avena sativa), pennywort (Centella asiatica), sun flower seed (Helianthus annuus), lemon balm (Melissa officinalis), and star anise (Illicium verum). In some embodiments, the compositions are prepared by a method comprising adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water, and heating the water to about 100° C. for about 45 minutes to prepare an aqueous extract; adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water to prepare a mixture, adding Lactobacillus sakei to the mixture, and fermenting the mixture at about 20° C. to prepare a fermented extract; and combining the aqueous extract and fermented extract (e.g., at a 1:1 ratio). In some embodiments, the compositions disclosed herein further comprise one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil and Vitamin E oil (gamma-tocopherol). In some embodiments, the compositions comprise about 10-12% cold-pressed oils, wherein the cold-pressed oils comprise a mixture of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

In some embodiments, the compositions disclosed herein are formulated as a paste, gel, cream, lotion, powder, soap, mask, or spray. In some embodiments, the compositions are formulated as a skin or hair care product. In some embodiments, the compositions are formulated for topical administration. In some embodiments, the compositions are formulated as a lotion or cream. In some embodiments, the combination of the aqueous extract and fermented extract are combined with a salt (e.g., magnesium sulfate, e.g., as a bath salt).

The above discussed, and many other features and attendant advantages of the present inventions will become better understood by reference to the following detailed description of the invention when taken in conjunction with the accompanying examples.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts an example schematic workflow showing a method of preparing an extract and fermentation with lactic acid bacteria and combining stages according to an embodiment of the invention.

FIGS. 2A-2D show photographic results from different subjects having psoriasis administered a cream formulation of the present invention, with before and after time points. LH, left hand; RH, right hand.

FIGS. 3A-3D show photographic results from different subjects having eczema administered a cream formulation of the present invention, with before and after time points. LH, left hand; RH, right hand.

FIGS. 4A-4B show photographic results from different subjects using a cream formulation of the present invention on dry, cracked skin, at different time points.

FIGS. 5A-5B show photographic results from different subjects administered a cream formulation of the present invention on UV radiation (sunburn) at different time points.

FIG. 6 shows photographic results from using a cream formulation of the present invention on soft tissue injury.

FIG. 7 shows photographic results from using a cream formulation of the present invention on a birth mark/mole at different time points.

FIG. 8 shows photographic results from using a cream formulation of the present invention on a planter's wart.

FIGS. 9A-9C show photographic results from different subjects using a cream formulation of the present invention on psoriasis at different time points.

FIG. 10 shows photographic results from using a cream formulation of the present invention on a skin rash.

FIGS. 11A-11C show photographic results from different subjects using a cream formulation of the present invention on acne at different time points.

FIGS. 12A-12C show photographic results from different subjects using a cream formulation of the present invention on scars from acne and a burn at different time points.

FIGS. 13A-13B show photographic results from different subjects using a cream formulation of the present invention on peeling and cracked skin at different time points.

FIGS. 14A-14B show photographic results from different subjects using a cream formulation of the present invention on seborrheic dermatitis.

### DETAILED DESCRIPTION OF THE INVENTION

The present invention relates to the preparation and use of botanical extracts, fermentations of the same, and combinations of the same with cold-pressed oils, in various formulations to treat multiple skin diseases/afflictions with inflammation in tissue injury and at different stages of inflammation. The present invention provides a natural solution to often painful, unsightly, embarrassing and irritating skin problems. The inventions generally relate to botanical extracts combined with cold-pressed oils in various formulations to treat skin disorders associated with itching (pruritus) and irritations, with immediate relief from itching. The present invention is effective on multiple stages of inflammation.

The invention provides a natural plant alternative for managing skin diseases/afflictions with inflammation and pruritus to the use of topical steroids or systemic use of biopharmaceuticals with their associated side effects. A problem addressed by the present invention when used for managing skin diseases/afflictions with inflammation and pruritus, is the absence of antibiotic use and need to reestablish the gut flora following antibiotic treatment. A further problem addressed by the present invention, is sleeplessness caused by itching, painful and irritated skin. The present invention provides both long term and immediate relief, enabling subjects having itching, painful and irritated skin to get to sleep or rest more quickly and for a longer duration.

To address the above problems, a botanical extract has been prepared and combined with cold-pressed oils in various formulations. Such compositions have synergistic activity to prevent inflammation and itching skin.

According to some embodiments of the invention, combining the botanical extract and cold-pressed oils as described herein have synergistic activity for the production of collagen, repair of damage to the lipid barrier of the epidermis, soothing irritation, reducing inflammation and improving skin tone.

Compositions

Some aspects of the invention are related to a composition comprising botanical extracts. In some embodiments, the composition comprises pomegranate fruit (Punica granatum) extract, oats (Avena sativa) extract, pennywort (Centella asiatica) extract, sun flower seed (Helianthus annuus) extract, lemon balm (Melissa officinalis) extract, and star anise (Illicium verum) extract. In some embodiments, the composition comprises an aqueous botanical extract that is prepared by combining by weight 0.001% to 10% (w/v) of pomegranate fruit (e.g., whole, powder, extract), 0.001% to 20% oats (e.g., rolled, groats, steel cut, oat bran, oat fiber), 0.001% to 5% of pennywort (e.g., dried herb, alcohol free extract, liquid extract, tea infusion), 0.001% to 2% of sunflower seeds (e.g., raw, powdered), 0.001% to 1% of lemon balm (e.g., powder, leaves, tea infusion), 0.001% to 1% of star anise (e.g., seeds, powder, tea infusion) with water, and heating the water to about 100□C for 45 minutes. In some embodiments, the composition comprises a fermented botanical extract that is prepared by combining by weight 0.001% to 10% (w/v) of pomegranate fruit (e.g., whole, powder, extract), 0.001% to 20% oats (e.g., rolled, groats, steel cut, oat bran, oat fiber), 0.001% to 5% of pennywort (e.g., dried herb, alcohol free extract, liquid extract, tea infusion), 0.001% to 2% of sunflower seeds (e.g., raw, powdered), 0.001% to 1% of lemon balm (e.g., powder, leaves, tea infusion), 0.001% to 1% of star anise (e.g., seeds, powder, tea infusion) with water to provide a mixture, and fermenting the mixture with lactic acid bacteria. In some embodiments, the composition comprises a mixture of aqueous botanical extract and fermented botanical extract.

In some embodiments, the composition comprising botanical extracts further comprises suitable ingredients for a bath salt. In some embodiments, the bath salt further comprises magnesium sulfate (Epsom salts), sodium chloride (table salt), sodium bicarbonate (baking soda), sodium hexametaphosphate (amorphous/glassy sodium metaphosphate), sodium sesquicarbonate, borax, or sodium citrate. In some embodiments, the bath salts comprise glycerin, or liquid glycerin. In some embodiments, the bath salts comprise one or more humectants, thickeners, or emulsifying agents (e.g., as described herein).

Some aspects of the invention are related to a composition comprising one or more botanical extracts and one or more cold-pressed oils, wherein (a) the one or more botanical extracts is selected from the group consisting of pomegranate fruit (Punica granatum) extract, oats (Avena sativa) extract, pennywort (Centella asiatica) extract, sun flower seed (Helianthus annuus) extract, lemon balm (Melissa officinalis) extract, and star anise (Illicium verum) extract; and (b) the one or more cold-pressed oils is selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

In some embodiments, pomegranate fruit extract refers to an extract from pomegranate fruit comprising one or more of ellagitannins, ellagic acid (and its derivative 3,8-dihydroxy-6H-dibenzo[b, d]pyran-6-one (urolithin A, UA)), luteolin, and punicic acid.

[1] In some embodiments, oats extract refers to an extract from oats comprising one or more of β-Glucan, avenanthramides, and avenacosides A and B, C (and metabolites 5-hydroxyanthranilic acid, dihydrocaffeic acid, caffeic acid, dihydroferulic acid, ferulic acid, dihydroavenanthramide-C, dihydroavenanthramide-B, and avenanthramide-B), alkylresorcinols, benzoxazinoids, flavonoids, lignans, and phytosterols.

In some embodiments, pennywort extract refers to an extract from pennywort comprising one or more of linalool, geraniol, alpha-terpinene, ketone, sesquiterpene and hydrocarbons such as beta-caryophyllene, humulene and alpha-copaene.

In some embodiments, sun flower seed extract refers to an extract from sun flower seeds comprising one or more triglycerides 50:2, 52:3, 52:4, 54:5, and 54:6; reproalbumin with sunflower trypsin inhibitor-1 cyclic peptide SFTI-1 (sunflower trypsin inhibitor-1); and heterodimeric 2S albumin.

In some embodiments, lemon balm extract refers to an extract from lemon balm comprising one or more flavonoids (rutin, myricetin, quercetin, kaempferol) and phenolic acids (gallic, p-coumaric, rosmarinic, syringic, caffeic, chlorogenic, ellagic, ferulic), caffeic acid derivatives (salvianolic acids, lithospermic acid) and rosmarinic acid derivatives (rosmarinic acid hexoside, sagerinic acid, sulfated rosmarinic acid).

In some embodiments, star anise extract refers to an extract from star anise comprising one or more trans-anethole, 2-(1-cyclopentenyl)-furan, cis-anethole, propiophenone, 4′-methoxy-propiophenone, and trans-β-methylstyrene.

As used herein, cold-pressed oil refers to oil prepared by applying mechanical pressure to press and collect oil from whole seeds or seed paste, while maintaining a temperature below 100° F. or 35° C., in order to prevent heat degradation of the oil components.

In some embodiments, the one or more botanical extracts are aqueous botanical extracts, fermented extracts, aqueous/alcoholic botanical extracts, alcoholic botanical extracts, organic solvent botanical extracts, enzymatic botanical extracts, acidic (e.g., vinegar) botanical extracts, pressure extracted botanical extracts, botanical extracts prepared by distillation, and/or botanical extracts prepared via saponification.

In some embodiments, the one or more botanical extracts comprise aqueous botanical extracts. In some embodiments, the aqueous botanical extracts are prepared by combining weight to volume (w/v) 0.001% to 10% of pomegranate fruit (e.g., whole, powder, or extract), 0.001% to 20% oats (e.g., rolled, groats, steel cut, oat bran, or oat fiber), 0.001% to 5% of pennywort (e.g., dried herb, alcohol free extract, liquid extract, or tea infusion), 0.001% to 2% of sunflower seeds (e.g., raw or powdered), 0.001% to 1% of lemon balm (e.g., powder, leaves, or tea infusion), 0.001% to 1% of star anise (e.g., seeds, powder, or tea infusion) with water, and heating the water to about 100□C for 45 minutes. In some embodiments, the water is heated prior to adding the botanical material. In some embodiments, the aqueous botanical extract is prepared by combining whole or powdered pomegranate fruit, oats, pennywort, sunflower seeds, lemon balm, and star anise with water, and heating the water to about 100□C for 45 minutes.

In some embodiments, the one or more botanical extracts comprise fermented botanical extracts. In some embodiments, the fermented botanical extracts are prepared by combining weight to volume (w/v) 0.001% to 10% of pomegranate fruit (e.g., whole, powder, or extract), 0.001% to 20% oats (e.g., rolled, groats, steel cut, oat bran, or oat fiber), 0.001% to 5% of pennywort (e.g., dried herb, alcohol free extract, liquid extract, or tea infusion), 0.001% to 2% of sunflower seeds (e.g., raw or powdered), 0.001% to 1% of lemon balm (e.g., powder, leaves, or tea infusion), 0.001% to 1% of star anise (e.g., seeds, powder, or tea infusion) with water to provide a mixture, and fermenting the mixture with lactic acid bacteria (e.g., Lactobacillus sakei) at about 20□C, optionally with mixing at about 45 rpm, for 24-42 hours. In some embodiments, the fermented botanical extract is prepared by combining whole or powdered pomegranate fruit, oats, pennywort, sunflower seeds, lemon balm, and star anise with water, and fermenting the mixture with lactic acid bacteria (e.g., Lactobacillus sakei) at about 20□C, optionally with mixing at about 45 rpm, for 24-42 hours.

In some embodiments, the composition comprises botanical extracts at a ratio of about 4:1, 3:1, 2:1, 1:1, 1:2, 1:3, or 1:4 of aqueous botanical extract to fermented botanical extract. In some embodiments, the composition comprises botanical extracts at a ratio of about 1:1 of aqueous botanical extract to fermented botanical extract.

In some embodiments, the composition comprises an emulsion comprising the one or more botanical extracts and the cold-pressed oils. In some embodiments, the composition further comprises an emulsification agent. In some embodiments, the emulsion is an oil-in-water emulsion. In some embodiments, the emulsion is a water-in-oil emulsion. In some embodiments, the emulsion may further comprise cosmetically acceptable ingredients. In some embodiments, the cosmetically acceptable ingredients comprise one or more solid oils, humectants, thickeners, and emulsifying agents.

Suitable solid oils for use in the present invention include, but are not limited to, shea butter, cocoa butter, avocado butter, aloe butter, coconut oil, coffee butter and palm oil.

Suitable humectants for use in the present invention include, but are not limited to, triethylene glycol, tripropylene glycol, propylene glycol, glycerin, sorbitol, urea and hyaluronic acid.

Suitable thickeners for use in the present invention include, but are not limited to, carrageen, cellulose gum, guar gum xanthan gum, bees wax, carnauba wax, glyceryl stearate and cetyl acetic acid (stearic acid).

Suitable emulsifying agents for use in the present invention include, but are not limited to, polyethylene glycol (PEG), beeswax, candelilla wax, lecithin, stearic acid, cetostearyl alcohol polysorbate 60 and glyceryl stearate citrate.

In some embodiments, the composition comprises at least two, at least three, at least four, at least five, or all six botanical extracts. In some embodiments, the composition comprises at least two, at least three, at least four, at least five, or all six botanical extracts as both aqueous botanical extracts and fermented botanical extracts.

In some embodiments, the emulsion comprises at least two, at least three, at least four, at least five, at least six, at least seven, at least eight, or all nine cold-pressed oils.

In some embodiments, the one or more botanical extracts have been subjected to fermentation. In some embodiments, the fermentation was lactic acid fermentation. In some embodiments, the lactic acid bacteria are selected from the group consisting of Lactobacillus sakei, Lactobacillus brevis and combinations thereof.

In some embodiments, the percentage (w/w) of botanical extract (BE) in the composition is about 99%, 95%, 90%, 85%, 80%, 75%, 70%, 65%, 60%, 55%, 50%, 45%, 40%, 35%, 30%, 25%, 20%, 15%, 10%, 5%, or 1%. In some embodiments, the percentage (w/w) of botanical extract in the composition is less than 1%, about 1%, about 5%, or is about 8.8%. In some embodiments, the percentage (w/w) of botanical extract in the composition is about 5% to 8.8%.

In some embodiments, the percentage (w/w) of cold-pressed oil (CPO) in the composition is about 99%, 95%, 90%, 85%, 80%, 75%, 70%, 65%, 60%, 55%, 50%, 45%, 40%, 35%, 30%, 25%, 20%, 15%, 10%, 5%, or 1%. In some embodiments, the percentage (w/w) of CPO in the composition is about 14%, or is about 12%. In some embodiments, the percentage (w/w) of CPO in the composition is about 10% to 20%.

In some embodiments, the percentage (w/w) of BE in the composition is about 2.5% to 8.8%, and the percentage (w/w) of CPO in the composition is about 10% to 20%. In some embodiments, the percentage (w/w) of BE in the composition is about 2.5% and the percentage (w/w) of CPO in the composition is about 12%. In some embodiments, the percentage (w/w) of BE in the composition is about 5% and the percentage (w/w) of CPO in the composition is about 12%. In some embodiments, the percentage (w/w) of BE in the composition is about 5% and the percentage (w/w) of CPO in the composition is about 14%. In some embodiments, the percentage (w/w) of BE in the composition is about 8.8% and the percentage (w/w) of CPO in the composition is about 12%. In some embodiments, the percentage (w/w) of BE in the composition is about 8% and the percentage (w/w) of CPO in the composition is about 14%.

In some embodiments, the composition further comprises pro-vitamin B5. In some embodiments, the pro-vitamin B5 is provided in tea tree (Melaleuca alternifolia) oil, jojoba (Simmondsia chinensis) oil, argan (Argania spinose) oil, and/or vitamin E oil. In some embodiments, the concentration of pro-vitamin B5 is about 5% (w/v). In some embodiments, the composition further comprises other substances known in the art to be useful for skin repair, including, for example, vitamin C, Zinc, hyaluronic acid, green tea extract, retinol, coenzyme Q10 or alpha-lipoic acid.

In some embodiments, the composition is prepared in any type of formulation generally accepted in this field, including a solution, suspension, emulsion, paste, gel, cream, lotion, powder, soap, surfactant-containing cleansing, oil, powdered foundation, emulsion foundation, waxed foundation and spray. In some embodiments, the composition is prepared as a skin and/or hair-care preparation. In some embodiments, the skin and/or hair-care preparation is a body cream, a face cream, a face mask, a lotion, a toner, an ointment; a gel, a spray; a hair shampoo, a hair lotion; a foam bath composition, a shower bath composition, bath soaking salts; a powder or a soap. In some embodiments, the composition is formulated for topical administration. In some embodiments, the composition is formulated as a lotion or cream.

In some embodiments, the composition is the composition provided by Example 1. In some embodiments, the composition is the composition provided by Example 2. In some embodiments, the composition is the composition provided by Example 3. In some embodiments, the composition is the composition provided by Example 4. In some embodiments, the composition is the composition provided by Example 5. In some embodiments, the composition is the composition provided by Example 6. In some embodiments, the composition is the composition provided by Example 7. In some embodiments, the composition is the composition provided by Example 8. In some embodiments, the composition is the composition provided by Example 9. In some embodiments, the composition is the composition provided by Example 10. In some embodiments, the composition is the composition provided by Example 11. In some embodiments, the composition is the composition provided by Example 12. In some embodiments, the composition is the composition provided by Example 13. In some embodiments, the composition is the composition provided by Example 14. In some embodiments, the composition is the composition provided by Example 15. In some embodiments, the composition is the composition provided by Example 16. In some embodiments, the composition is the composition provided by Example 17. In some embodiments, the composition is the composition provided by Example 18. In some embodiments, the composition is the composition provided by Example 19. In some embodiments, the composition is the composition provided by Example 20. In some embodiments, the composition is the composition provided by Example 21. In some embodiments, the composition is the composition provided by Example 22. In some embodiments, the composition is the composition provided by Example 23. In some embodiments, the composition is the composition provided by Example 24. In some embodiments, the composition is the composition provided by Example 25. In some embodiments, the composition is the composition provided by Example 26. In some embodiments, the composition is the composition provided by Example 27. In some embodiments, the composition is the composition provided by Example 28. In some embodiments, the composition is the composition provided by Example 29. In some embodiments, the composition is the composition provided by Example 30. In some embodiments, the composition is the composition provided by Example 31. In some embodiments, the composition is the composition provided by Example 32. In some embodiments, the composition is the composition provided by Example 33. In some embodiments, the composition is the composition provided by Example 34. In some embodiments, the composition is the composition provided by Example 35.

Methods of Manufacture

Some aspects of the present invention are related to a method of manufacturing the compositions disclosed herein. (a) obtaining one or more aqueous botanical extracts by heating an aqueous solution comprising botanical material from one or more plants selected from pomegranate fruit (Punica granatum), oats (Avena sativa), pennywort (Centella asiatica), sun flower seed (Helianthus annuus), lemon balm (Melissa officinalis), and star anise (Illicium verum); (b) obtaining one or more fermented botanical extracts by fermenting an aqueous solution comprising botanical material from one or more plants selected from pomegranate fruit, oats, pennywort, sun flower seed, lemon balm, and star anise; (c) removing insoluble material from the one or more botanical extracts obtained in steps (a) and (b) and combining the resultant extracts, or combining the botanical extracts of (a) and (b) and removing insoluble material from the combined botanical extract; and (d) emulsifying the product of step (c) with one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil and Vitamin E oil (gamma-tocopherol). In some embodiments, the aqueous botanical extracts are prepared in step (a) by combining 0.001% to 10% (w/v) of pomegranate fruit (e.g., whole, powder, or extract), 0.001% to 20% (w/v) oats (e.g., rolled, groats, steel cut, oat bran, or oat fiber), 0.001% to 5% (w/v) of pennywort (e.g., dried herb, alcohol free extract, liquid extract, or tea infusion), 0.001% to 2% (w/v) of sunflower seeds (e.g., raw or powdered), 0.001% to 1% of lemon balm (w/v) (e.g., powder, leaves, or tea infusion), 0.001% to 1% of star anise (w/v) (e.g., seeds, powder, or tea infusion) with water, and heating the water to about 100□C for 45 minutes. In some embodiments, the water is heated prior to adding the botanical material. In some embodiments, the aqueous botanical extract is prepared by combining whole or powdered pomegranate fruit, oats, pennywort, sunflower seeds, lemon balm, and star anise with water, and heating the water to about 100□C for 45 minutes.

In some embodiments of step (b), one or more fermented botanical extracts is obtained by fermenting an aqueous solution comprising botanical material from one or more plants selected from pomegranate fruit (Punica granatum), oats (Avena sativa), pennywort (Centella asiatica), sun flower seed (Helianthus annuus), lemon balm (Melissa officinalis), and star anise (Illicium verum) with lactic acid bacteria for a suitable time (e.g., 20-24° C. for about 36-72 hours), followed by heat inactivation of the bacteria (e.g., at 56° C. for about 30 minutes or more).

In some embodiments, the botanical extracts according to the invention may be present as water-containing preparations and/or as preparation dissolved in organic solvents, spray dried or freeze-dried water-free solids, a fermented sample as whole or distilled, alcoholic or aqueous/alcoholic extraction, with protease enzymes, or with vinegar. In some embodiments, the botanical extract according to the invention may be freeze-dried, lyophilized, and/or made into a powder. In some embodiments, the botanical extracts according to the invention may be concentrated. In some embodiments, the botanical extracts are concentrated by evaporation. In some embodiments, the botanical extracts are concentrated by at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more. In some embodiments, the botanical extracts are concentrated by about 16-fold to 17-fold. In some embodiments, each 50 ml of extract is concentrated to a volume of 3 ml (i.e., concentrated by 16.67-fold). In some embodiments, the botanical extract according to the invention is used in quantities of 0.001 to 8.8% by volume and mixed with quantities in the particular formulation that add up to 100% by weight and optionally other auxiliaries and additives.

In some embodiments, insoluble material (e.g., insoluble fruit, oats, plant fiber, seeds, or leaf material) is removed in step (c) by filtration. In some embodiments, insoluble material is removed in step (c) by centrifugation.

In some embodiments, step (c) further comprises emulsifying the aqueous botanical extract after removal of insoluble material with one or more cosmetically acceptable ingredients. In some embodiments, the cosmetically acceptable ingredients comprise one or more solid oils, humectants, thickeners, and emulsifying agents, as described herein.

In some embodiments, the fermented botanical extracts of step (b) is fermented. In some embodiments, the product of fermentation is distilled, filtered, or fractionated. In some embodiments, the fermentation is with lactic acid bacteria. In some embodiments, the lactic acid bacteria are selected from the group consisting of Lactobacillus sakei, Lactobacillus brevis, and combinations thereof. In some embodiments, fermentation (e.g., fermentation with lactic acid bacteria) is performed at about 20-24° C. for about 36 to 72 hours. In some embodiments, the fermentation is for about 42 hours.

In some embodiments, the fermentation is stopped by heat inactivation (e.g., heating the solution to kill or inactivate the fermenting micro-organism, e.g., at 56° C. for about 30 minutes or more).

In some embodiments, the one or more aqueous botanical extracts of step (a) are heated, or also heated, to degrade or inhibit non-heat stable components of the condensate. In some embodiments, the aqueous botanical extracts are heated to more than 40° C.

In some embodiments, the aqueous and/or fermented botanical extracts are cooled to less than about 40° C. prior to emulsification with cold-pressed oil. In some embodiments, the aqueous and/or fermented botanical extracts are cooled to room temperature prior to emulsification with cold-pressed oil.

In some embodiments of step (b), one or more fermented botanical extracts is obtained by fermenting an aqueous solution comprising botanical material from one or more plants selected from pomegranate fruit (Punica granatum), oats (Avena sativa), pennywort (Centella asiatica), sun flower seed (Helianthus annuus), lemon balm (Melissa officinalis), and star anise (Illicium verum) with lactic acid bacteria for a suitable time (e.g., 20-24° C. for about 36-72 hours), followed by heat inactivation of the bacteria (e.g., at 56° C. for about 30 minutes or more).

In some embodiments, the cold-pressed oils described herein are preferably used in quantities of 0.001 to 20% by volume and mixed with quantities in the particular formulation that add up to 100% by weight and optionally other auxiliaries and additives. From 0.001% to 1% of aloe vera oil (Barbados aloe), 0.001% to 1% rosehip oil (Rosa canina, R. gallica, R. rugosa, R. villosa). 0.001% to 3% Sea buckthorn seed oil (Flippophae rhamnoides), 0.001% to 1% pumpkin seed oil (Cucurbita pepo), 0.001% to 1% pomegranate seed oil (Tunica granatum), 0.001% to 1% Carrot seed oil (Daucus carota), 0.001% to 1% Chamomile oil (Matricaria chamomilla), 0.001% to 3% Coffee bean oil (Matricaria chamomilla). 0.001% to 1% raspberry seed oil (Rubus idaeus), and 0.001 to 4% Vitamin E oil (gamma-tocopherol, alpha-tocopherol).

In some embodiments, provitamin B5 is added to the one or more aqueous botanical extracts prior to step (c). In some embodiments, provitamin B5 is added prior to emulsification with cosmetically acceptable ingredients. In some embodiments, tea tree (Melaleuca alternifolia) oil, jojoba (Simmondsia chinensis) oil, argan (Argania spinose) oil, and/or vitamin E oil (e.g., (gamma-tocopherol and/or alpha-tocopherol) are added to the liquid oil components. In some embodiments, the amount of pro-vitamin B5 added is about 2% (w/v).

In some embodiments, the method further comprises formulating the composition into a cosmetic or skin care formulation. The composition may be formulated a solution, suspension, emulsion, paste, gel, cream, lotion, powder, soap, surfactant-containing cleansing, oil, powdered foundation, emulsion foundation, waxed foundation and spray. In some embodiments, the method comprises formulating the composition as a skin and/or hair-care preparation. In some embodiments, the skin and/or hair-care preparation is a body cream, a face cream, a face mask, a lotion, a toner, an ointment; a gel, a spray; a hair shampoo, a hair lotion; a foam bath composition, a shower bath composition, bath soaking salts; a powder or a soap. In some embodiments, the method comprises formulating the composition for topical administration. In some embodiments, the method comprises formulating the composition as a lotion or cream.

Some aspects of the disclosure are directed to a method of manufacturing the compositions described herein comprising the following steps: (a) providing one or more botanical extracts as disclosed herein; (b) removing insoluble material from the one or more botanical extracts obtained in step (a); and (c) emulsifying the product of step (b) with one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil and Vitamin E oil (gamma-tocopherol).

In some embodiments, the one or more botanicals extract of step (a) are prepared by aqueous extraction, fermentation, aqueous/alcoholic extraction, alcoholic extraction, organic solvent extraction, enzymatic extraction, acidic (e.g., vinegar) extraction, pressure extraction, extraction via distillation, and/or extraction via saponification.

Some aspects of the disclosure are directed to a method of manufacturing the compositions described herein comprising the following steps: (a) providing one or more botanical extracts as disclosed herein; (b) removing insoluble material from the one or more botanical extracts obtained in step (a); and formulating the product of step (b) as a body cream, a face cream, a face mask, a lotion, a toner, an ointment; a gel, a spray; a hair shampoo, a hair lotion; a foam bath composition, a shower bath composition, bath soaking salts; a powder or a soap.

In some embodiments, the method further comprises adding suitable ingredients to the product of step (b) for a bath salt. In some embodiments, the suitable ingredients comprise magnesium sulfate (Epsom salts), sodium chloride (table salt), sodium bicarbonate (baking soda), sodium hexametaphosphate (amorphous/glassy sodium metaphosphate), sodium sesquicarbonate, borax, or sodium citrate. In some embodiments, the suitable ingredients comprise glycerin, or liquid glycerin. In some embodiments, the bath salts comprise one or more humectants, thickeners, or emulsifying agents (e.g., as described herein). In some embodiments, the composition is dried (e.g, at 90 □C for 8-12 hours).

Some aspects of the present disclosure are related to a composition comprising extracts of pomegranate fruit (Punica granatum), oats (Avena sativa), pennywort (Centella asiatica), sun flower seed (Helianthus annuus), lemon balm (Melissa officinalis), and star anise (Illicium verum). In some embodiments, the composition is prepared by a method comprising adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water, and heating the water to 100□C for 45 minutes to prepare an aqueous extract; adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water to prepare a mixture, adding Lactobacillus sakei to the mixture, and fermenting the mixture at 20□C to prepare a fermented extract; and combining the aqueous extract and fermented extract. In some embodiments, the combination of the aqueous extract and fermented extract (e.g., at a ratio of about 1:1) are combined with a salt (e.g., magnesium sulfate). In some embodiments, the combination of the aqueous extract and fermented extract (e.g., at a ratio of about 1:1) are formulated as a body cream, a face cream, a face mask, a lotion, a toner, an ointment; a gel, a spray; a hair shampoo, a hair lotion; a foam bath composition, a shower bath composition, bath soaking salts; a powder or a soap.

In some embodiments, the method is the method provided by Example 1. In some embodiments, the method is the method provided by Example 2.

Methods of Treatment

Some aspects of the present disclosure are directed to using the compositions described herein for treating a subject having a skin disease, disorder or condition. In some embodiments, the composition is administered topically to the subject. In some embodiments, the composition of Example 1 or Example 2 is administered.

In some embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples.

In some embodiments, the skin disease, disorder or condition is selected from the group consisting of acne, athlete's foot, bed sore, boil, calluses and corns, canker sore, carbuncles, candidiasis (e.g., oral (oral thrush), vaginal (candidal vulvovaginitis), penile (candidal balanitis), in the diaper area (diaper rash), in the skin folds (candidal intertrigo), cellulitis, cold sores, creeping eruption, dermatitis (eczema) (e.g., atopic dermatitis, contact dermatitis, seborrhoeic dermatitis, cradle cap, nummular dermatitis, stasis dermatitis, perioral dermatitis (muzzle rash), dermatitis herpetiformis), dermatofibroma, eczema, friction blisters, herpes (e.g., HHV1 i.e., cold sores, HHV2 i.e., genital herpes, HHV3, e.g., chickenpox, shingles, HHV6, HHV7, e.g., roseola infantum, sixth disease, HHV8, i.e., Kaposi's sarcoma herpesvirus), hidradenitis suppurativa, hives, ichthyosis, impetigo, jock itch, Kaposi's sarcoma, keratoacanthoma, keratosis (e.g., actinic (solar) keratosis, keratosis pilaris, keratosis follicularis (Darrier's disease), seborrheic, and hyperkeratosis), lice infection, lichen planus, lichen simplex chronicus, malignant melanoma, miliaria, molluscum contagiosum, pediculosis, pemphigus, photoallergy, photosensitivity, Pityriasis rosea, Pityriasis rubra, pilaris, psoriasis, ring worm, Saint Anthony's fire, scabies, scaring (e.g. hypertrophic, keloid, acne, burns, abrasions, stretch marks (growth, weight loss/gain), tattoos, tattoo removal), scleroderma, sebaceous cyst, shingles, skin cancer, skin tags, tick bite, tinea: (barbae, capitis, corporis, cruris (Jock Itch), pedis unguium, versicolor), trichomycosis, vitiligo, or warts (e.g., common, planar, genital). In some embodiments, the skin disease, disorder or condition is a skin inflammatory disease. In some embodiments, the skin inflammatory disease is selected from the group consisting of acne, dermatitis, eczema, oily skin, rosacea, cutaneous lymphoma and urticaria. In some embodiments, the dermatitis is selected from the group consisting of atopic dermatitis, psoriasis and contact dermatitis.

In some embodiments, the skin disease, disorder or condition is selected from the group consisting of hidradenitis suppurativa (HS) acne inversa, psoriasis, eczema, dry skin, sunburn, soft tissue injury, insect bite, radiation dermatitis, birth marks and moles, planter's wart, skin rash, acne vulgaris, dermal scaring and keloids from acne, dermal scaring and keloids from cutaneous burn injuries, skin peeling and cracking, and seborrheic dermatitis.

The composition according to the present invention may be administered at specific fixed or variable intervals, e.g., once a day, or on an “as needed” basis. The composition according to the present invention may be administered in a dose range varying depending on the surface area of the affected skin, age, gender, health condition, administration time, administration method, excretion rate and disease severity.

The composition according to the present invention can be administered 1, 2, 3, 4, 5, 6, 7, 8, 9 or 10 times per day. Preferably, the composition according to the present invention is administered once per day.

The composition according to the present invention can be administered regularly for long periods of time. In an embodiment, the composition can be administered regularly for 1, 2, 3, 4, 5, 6, 7, 8, 9, 10 or more years. In another embodiment, the composition can be administered regularly for 1, 2, 3, 4, 5, 6, 7, 8, 9, 10, 11, 12 or more months. In other embodiments, the composition can be administered regularly for 1, 2, 3, 4, 5, 6, 7, 8, 9, 10 or more weeks. As used herein, the term “regularly” refers to administration of the composition at regular times or intervals over a period of time. For instance, the composition may be administered to a patient once daily for 1 month. In other embodiments, the composition may be administered to a patient once daily for 1 week. In other embodiments, the composition may be administered to a patient once daily for 1 year. It should be appreciated that the frequency of administration may vary based on a number of factors, including, but not limited to, the severity of disease, the overall health of the patient, any additional medications the patient is taking, and whether the treatment is prophylactic or not. It should also be appreciated that the frequency of administration may be adjusted at any point.

As used herein, “treat,” “treatment,” “treating,” or “amelioration” when used in reference to a skin disease, disorder or condition, refer to therapeutic treatments for a skin disease, disorder or condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment. In some embodiments, beneficial or desired clinical results include a reduction or elimination of itching. In some embodiments, beneficial or desired clinical results include a reduction or elimination of skin inflammation. In some embodiments, beneficial or desired clinical results include a reduction or elimination of flaking, burning, dryness, blisters, boils, psoriatic lesions, and/or prickling.

As used herein, the term “administering,” refers to the topical placement of the composition as disclosed herein by a method or route which results in delivery to a site of action. The composition can be administered by any appropriate route which results in an effective treatment in the subject.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

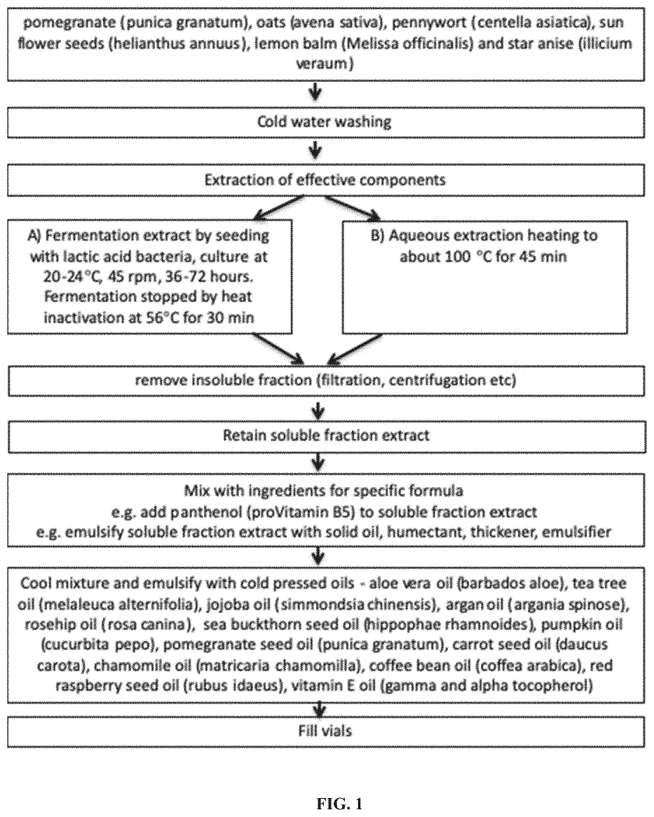

A fermented botanical extract was prepared as shown in FIG. 1 by combining and washing 200 g steel cut oats (Avena sativa), 50 g dried pennywort (Centella asiatica), 20 g raw sun flower seed (Helianthus annuus), 10 g dried lemon balm leaves (Melissa officinalis), and 10 g star anise seeds (Illicium verum) then adding 100 g pomegranate whole fruit powder (Punica granatum) and 3.5 liters of filtered water, and then seeding with 20 g Lactobacillus sakei, fermenting at 20□C, with mixing at 45 rpm, for 42 hours. Fermentation was stopped by heating to 56□C for 30 min (A in FIG. 1), and concentrated to 350 mL by heat evaporation.

An aqueous botanical extract was prepared as shown in FIG. 1 by combining and washing 200 g steel cut oats (Avena sativa), 50 g dried pennywort (Centella asiatica), 20 g raw sun flower seed (Helianthus annuus), 10 g dried lemon balm leaves (Melissa officinalis), and 10 g star anise seeds (Illicium verum) then adding 100 g pomegranate whole fruit powder (Punica granatum) and 3.5 liters of filtered water and then B) heating to 100□C for 45 minutes (B in FIG. 1), and concentrated to 350 mL by heat evaporation.

The extracts from fermentation and aqueous heating were filtered separately to remove solids and the extracts allowed to cool. Further insoluble material was filtered to separate and retain the soluble botanical extract.

To prepare 5% BE of 5% BE/12% CPO cream formulation, 50 mL of total botanical extract (referred to in the below examples as “BE”) was prepared by combining 1 volume of fermented botanical extract and 1 volume of aqueous botanical extract. 50 g pro-vitamin B5 and 50 g vitamin C was then dissolved in the 50 mL combined botanical extract and 450 mL of filtered water (45% water). A solid oil phase was prepared by melting 150 g shea butter (Vitellaria paradoxa) (15%), 30 mL glycerin, 30 g stearic acid and 70 g emulsifier wax (cetostearyl alcohol polysorbate 60) (e). A water and oil emulsion was prepared by mixing the entire volume of the prepared extract into the entire prepared solid oil phase, emulsifying for 3 min, and allowing to cool to 45□C (f).

To prepare 12% CPO of 5% BE/12% CPO cream formulation, the cold-pressed liquid oil components (g) were prepared by combining 5 mL aloe vera (barbados aloe) oil, 5 mL tea tree oil (Melaleuca alternifolia), 5 mL jojoba oil (Simmondsia chinensis), 10 mL argan oil (Argania spinose), 8 mL rosehip (Rosa canina) oil, 20 mL sea buckthorn seed (Hippophae rhamnoides) oil, 10 mL pumpkin (Cucurbita pepo) oil, 4 mL pomegranate seed (Punica granatum) oil, 10 mL carrot seed (Daucus carota) oil, 8 mL chamomile (Matricaria chamomilla) oil, 20 mL coffee bean (Coffea arabica) oil, 10 mL raspberry seed (Rubus idaeus) oil and 5 mL Vitamin E oil (gamma-tocopherol).

To prepare 5% BE/12% CPO cream formulation, the entire volume of cold-pressed oils (g) were added to the entire volume of the water and oil emulsion (f), emulsified for 3 min, and allowed to cool to room temperature.

### Example 2

A fermented botanical extract was prepared as shown in FIG. 1 by combining and washing 200 g steel cut oats (Avena sativa), 50 g dried pennywort (Centella asiatica), 20 g raw sun flower seed (Helianthus annuus), 10 g dried lemon balm leaves (Melissa officinalis), and 10 g star anise seeds (Illicium verum) then adding 100 g pomegranate whole fruit powder (Punica granatum) and 3.5 liters of filtered water and then A) seeding with 20 g Lactobacillus sakei, fermenting at 20□C, with mixing at 45 rpm, for 42 hours. Fermentation was stopped by heating to 56□C for 30 min, and concentrated to 350 mL by heat evaporation.

An aqueous botanical extract was prepared as shown in FIG. 1 by combining and washing 200 g steel cut oats (Avena sativa), 50 g dried pennywort (Centella asiatica), 20 g raw sun flower seed (Helianthus annuus), 10 g dried lemon balm leaves (Melissa officinalis), and 10 g star anise seeds (Illicium verum) then adding 100 g pomegranate whole fruit powder (Punica granatum) and 3.5 liters of filtered water and then B) heated to 100□C for 45 minutes, and concentrated to 350 mL by heat evaporation.

The fermented and aqueous extracts were filtered separately to remove solids and the extracts allowed to cool. Further insoluble material was filtered to separate and retain the soluble botanical extract.

To prepare the bath salts formulation, 50 mL of botanical extract was prepared by combining 1 volume of fermented extract and 1 volume of aqueous heat extract. The combined botanical extract was concentrated to 3 mL by heat evaporation and mixed with 1800 g magnesium sulfate. The mixture was dried overnight at 90□C.

### Example 3

An approximately 37 year old female, with diagnosed Hidradenitis suppurativa (HS) acne inversa, having multiple surgeries to remove infected boils and tissue, in the groin and breast areas and with high frequency use of antibiotics and associated disruption of microbiome. She used a bath salts formulation of Example 2 for a period of 2 years, noticing that the boils softened and reduced then disappeared, had no surgeries or use of antibiotics during this time, her microbiome was undisturbed enabling her to work and live normally using a natural product to maintain her condition.

### Example 4

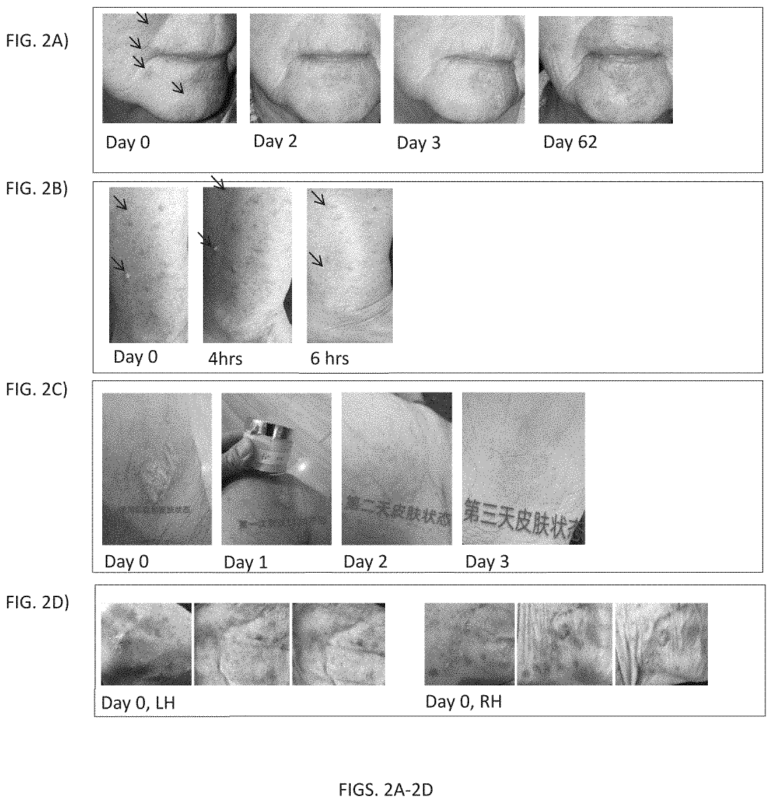

An 81 year old female, with diagnosed psoriasis, and using Clobetasol propionate cream, began using different percentages of BE/cold-pressed oil (CPO) (8% BE/14% CPO, 5% BE/12% CPO, 2.5% BE/12% CPO) cream formulation of Example 1 at different times over a year, daily on her face. With all percentages of BE/CPO used she reported the immediate caseation of itching, the lesions reduced over days of treatment, and the diminished use of makeup concealer and embarrassment associated with the plaques (FIG. 2A).

The same subject has psoriasis on her leg with characteristic silvery-white scaling and pruritic skin plaques (FIG. 2B). After treatment with different percentages of BE/CPO, at different times, (5% BE/12% CPO, 2.5% BE/12% CPO) cream formulation of Example 1 resulted in the flaky appearance and inflammation of the plaque lesions being reduced over 4-6 hours, significantly reducing the urge to pick at the skin and itch the lesions.

### Example 5

An approximately 40 year old male, with psoriasis on his leg with the silvery-white scaling characteristic of the disease, started a (5% BE/12% CPO) cream formulation treatment prepared by the method of Example 1. After 3 days, the characteristic white, flaky, built up lesions were reduced to normal skin (FIG. 2C).

### Example 6

A 79 year old female, with diagnosed psoriasis, using a (5% BE/12% CPO) cream formulation prepared by the method of Example 1 for psoriatic lesions on hands and legs reported the itching sensation associated with psoriasis stopped immediately after application of the cream formulation. With daily use, the silvery white thick, flaky and scaly skin patches reduced to the basal skin level with healthy vascularized, pink skin tissue being revealed. The embarrassing scaly, white skin plaques on the hands was removed (FIG. 2D).

### Example 7

A 23 year old female with psoriasis and with familial history, began using a (8% BE/12% CPO) cream formulation prepared by the method of Example 1 on her arms. She found that the spread of the lesions halted and the associated itchiness stopped.

### Example 8

A 47 year old female with psoriasis since she was in her 20's using steroid cream to maintain her condition. She also has chronic dermatitis of her ear canal that is extremely itchy, wakes her up at night and has so much silver scales that sometime muffle her hearing. Visits to the ENT usually vacuum out the scales and prescribe creams After using both a 2.5% BE/12% CPO and a 5% BE/12% CPO cream formulation prepared by the method of Example 1, she experienced immediate alleviation of the burning and prickly, flaking in the T-zone of her face and some help with plaques on her temples.

### Example 9

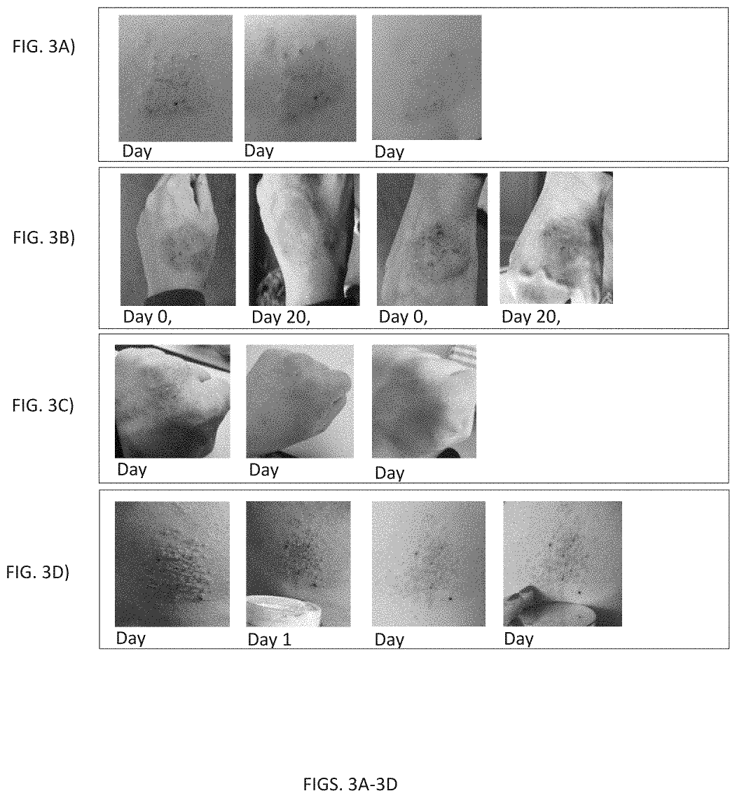

An approximate 40 year old male, with eczema on elbow (FIG. 3A), knuckles (FIG. 3C) and torso (FIG. 3D) began use of a (5% BE/12% CPO) cream formulation prepared by the method of Example 1, and the skin condition improved from flaky, dry and blistered eruptions to a smoother skin appearance.

### Example 10

An approximate 50 year old male, with diagnosed eczema on both hands (FIG. 3B) was using topical corticosteroid cream with little improvement to the skin condition for more than 10 years. After 20 days of using a (5% BE/12% CPO) cream formulation prepared by the method of Example 1 twice daily, the skin appears less irritated, softer and less inflamed. In a blinded study he also applied the base cream alone to one hand and the 5% BE/12% CPO cream formulation prepared by the method of Example 1 to the other hand and could tell the difference immediately. No relief and effect resulted from the base cream on one hand, while the 5% BE/12% CPO cream formulation prepared by the method of Example 1 on the other hand responded. He appreciates that he can use the cream formulation as much as needed without worrying about side effects and having to monitor the stop and start regime when using topical steroids.

### Example 11

An approximate 48 year old man, with chemical induced eczema on both arms and used topical corticosteroid cream to manage the intense burning and prickling of affected areas. He began using a (8% BE/12% CPO) cream formulation prepared by the method of Example 1 and had immediate relief from the intense burning and his skin retained more moisture.

### Example 12

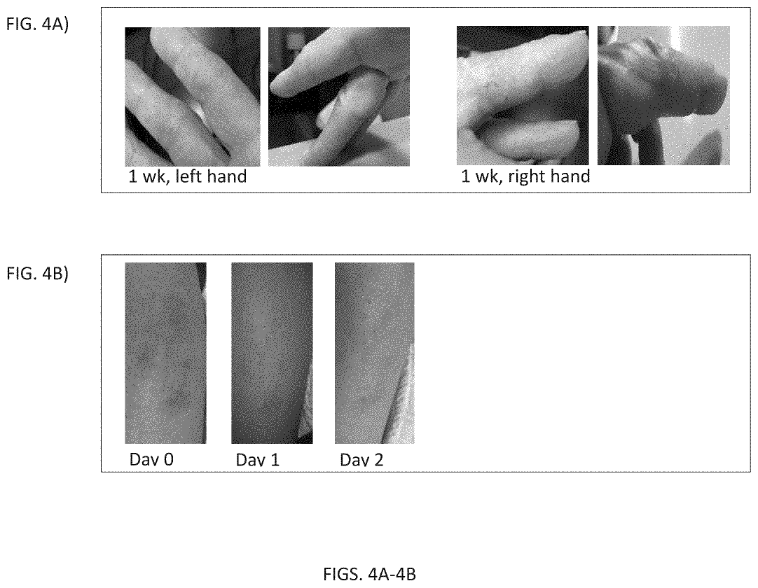

An approximate 68 year old female, with severely dry and cracked skin on her fingers and hands that she picked at constantly due to them being irritated and sore applied the (5% BE/12% CPO) cream formulation prepared by the method of Example 1 daily. After 1 week her condition was the best it has ever been (FIG. 4A).

### Example 13

A 14 year old male, with dry irritated skin on his lower leg, used a (5% BE/12% CPO) cream formulation prepared by the method of Example 1 that improved the appearance of his skin.

### Example 14

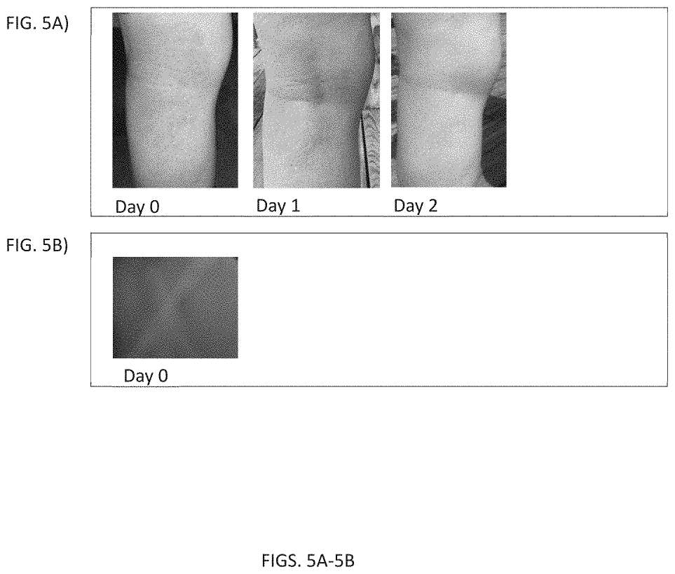

A 59 year old female with sunburn on the back of her legs (FIG. 5A), applied a (5% BE/12% CPO) cream formulation prepared by the method of Example 1 for 2 days. Experienced no pain, was able to sleep normally and go about her day without any discomfort.

### Example 15

A 52 year old female with sunburn on her back (FIG. 5B), applied a (5% BE/12% CPO) cream formulation prepared by the method of Example 1 for 2 consecutive days. Such a skin burn would have prevented her sleeping on her back, clothes would have irritated her skin and application of gels and lotions would have amplified the pain by resulting in sticking to clothing. With the application of the cream, she was able to wear regular clothes, sleep on her back. The lack of pain and heat after the skin burn were surprising and the lotion was not sticky.

### Example 16

A 55 year old female with sunburn on legs applied a (5% BE/12% CPO) cream formulation prepared by the method of Example 1 for 2 days after the sunburn. The next day the redness and dry, flaky skin were gone, the skin appearance was good and the cream acted as a humectant, retaining moisture of the skin.

### Example 17

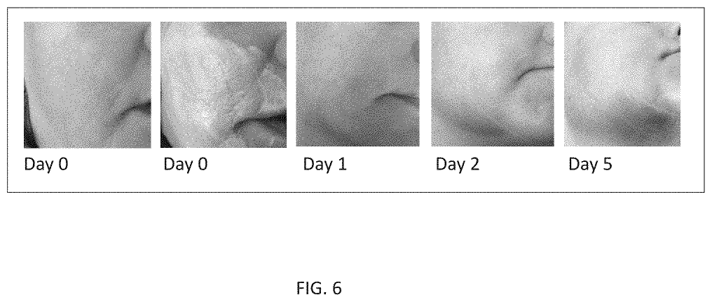

A 55 year old female after a soft tissue injury from gum surgery located on the lower and upper jaw, applied ice on and off, then applied a (5% BE/12% CPO) cream formulation prepared by the method of Example 1 overnight on day 0 (FIG. 6). The day after the surgery, she stopped all pain medication and experienced slight inflammation and, more importantly, no pain. Bruising was minimal, appearing on the third day post-surgery.

### Example 18

A 53 year old male who reacts strongly to insect bites, had multiple insect bites, that were itchy and irritating, applied a (8% BE/12% CPO) cream formulation prepared by the method of Example 1 resulting in no itching and the skin repaired itself faster than using corticosteroid or anti-histamine creams.

### Example 19

A 53 year old female with radiation dermatitis skin burn, after surgically removal of the breast tumor, used a (5% BE/12% CPO) cream formulation prepared by the method of Example 1 before sleeping. She got relief from the persistent heat, itch and angry skin resulting from the irradiation sufficient to be able to sleep.

### Example 20

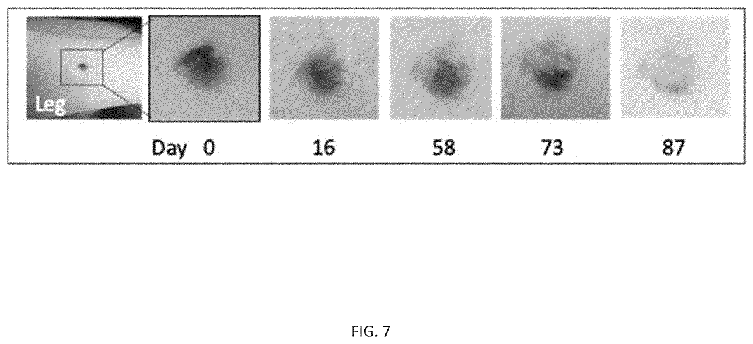

A 69 year old male with a birth mark/mole that was visibly changing and possibly pre-cancerous applied a (5.4% BE/12% CPO) cream formulation prepared by the method of Example 1 twice a day, morning and evening, for 16 days and then stopped. Visible changes occurred over time, the pigmented areas became lighter, anti-melanogenesis, and the lesion flattened for up to 87 days (FIG. 7).

### Example 21

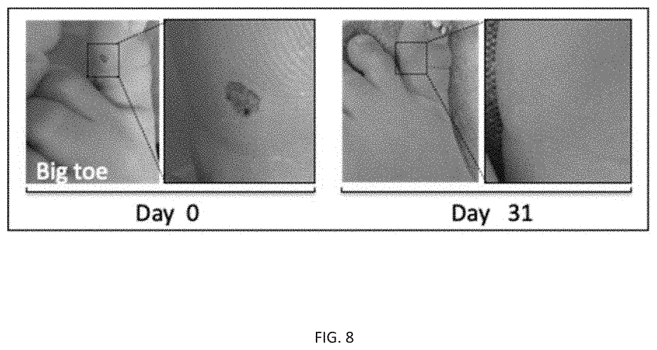

A 7 year old female with a planter's wart on the inside of the large toe (FIG. 8), which was swollen and at a painful location when wearing shoes. A cream formulation prepared by the method of Example 1 wherein the CPO was omitted, and having a BE concentration of between 0.9% and 1% was applied daily for one month resulting in the wart disappearing and normal skin in place of it.

### Example 22

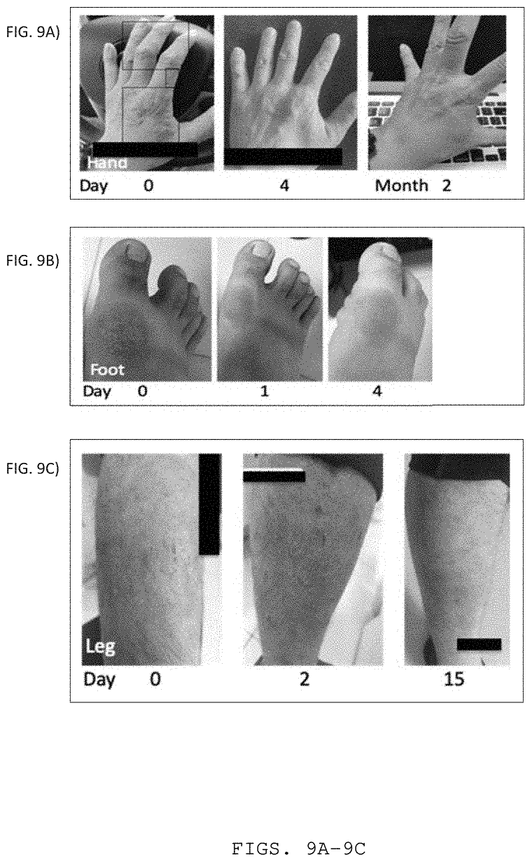

An approximately 40 year old male, with chronic psoriasis on his hand with the characteristic silvery-white scaling, blisters and bleeding cracks upon making a fist, started a (8.8% BE/12% CPO) cream formulation treatment prepared by the method of Example 1, applying twice a day. After 4 days, the characteristic blistering and white flaky built up lesions were reduced to normal skin and were maintained after 2 months with no bleeding or cracking when forming a first (FIG. 9A).

### Example 23

An approximately 40 year old male, with psoriasis on his foot with the silvery-white scaling characteristic of the disease, started a (8.8% BE/12% CPO) cream formulation treatment prepared by the method of Example 1, applying it daily. After 1 day, the characteristic itching and white, flaky, built up lesions were reduced, and by day 4 the skin was much improved (FIG. 9B).

### Example 24

An approximately 45 year old male, with psoriasis on the back of his leg with the silvery-white scaling and itchiness characteristic of the disease, started a (8.8% BE/12% CPO) cream formulation treatment prepared by the method of Example 1, applying it daily. After 15 days, the characteristic itchiness was gone and the white, flaky, built up lesions were reduced to normal skin (FIG. 9C).

### Example 25

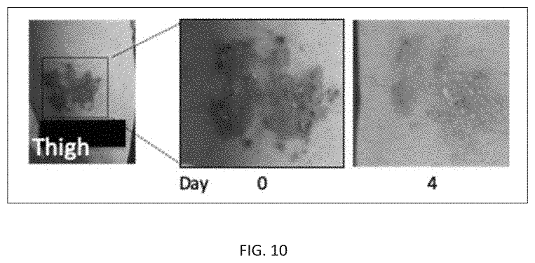

An approximately 40 year old male, with a skin rash, itchy red patches, started a (8% BE/12% CPO) cream formulation treatment prepared by the method of Example 1, applying it daily. After 4 days, the itching and red patches had reduced to a normal appearing skin (FIG. 10).

### Example 26

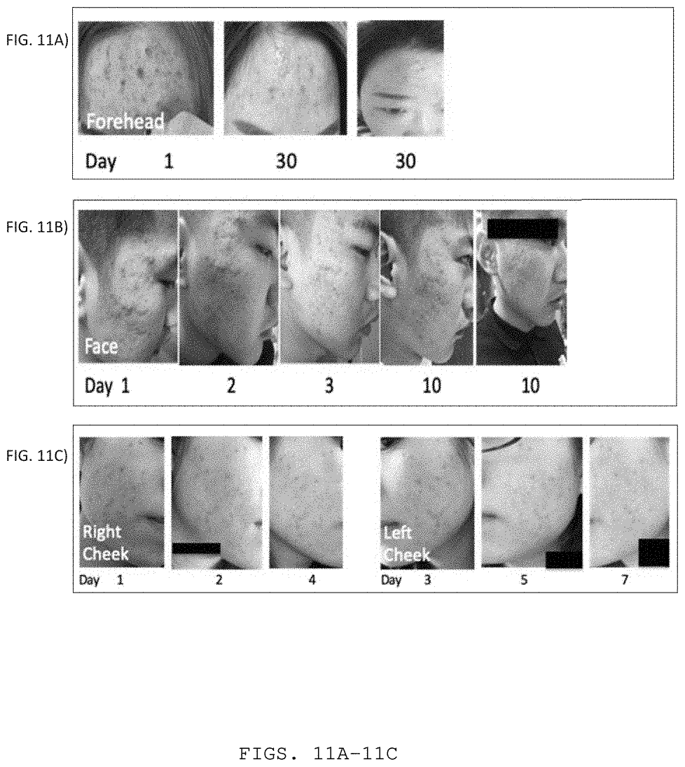

An approximately 20 year old female, with acne on her forehead, started a (8.8% BE) cream formulation treatment prepared by the method of Example 1 wherein the CPO was omitted, applying it twice daily. After 30 days, the red inflamed and pimply skin were all reduced (FIG. 11A).

### Example 27

An approximately 20 year old male, with acne on his face, started a (8.8% BE) cream formulation treatment prepared by the method of Example 1 wherein the CPO was omitted, applying it twice daily. After 3 days, the red inflamed and pimply skin were all reduced to near normal skin and improved further after 10 days (FIG. 11B).

### Example 28

An approximately 20 year old female, with acne on her face, started a (8.8% BE) cream formulation treatment prepared by the method of Example 1 wherein the CPO was omitted, applying it twice daily. After 4 (right cheek) and 7 (left cheek) days, the red blotchy and inflamed skin were reduced to smoother skin (FIG. 11C).

### Example 29

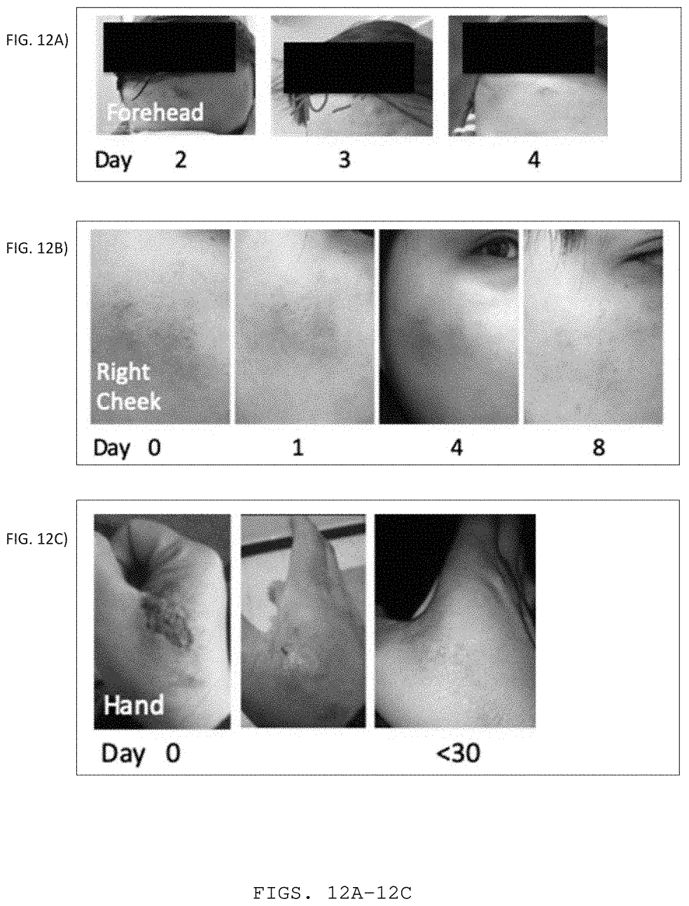

An approximately 25 year old male, with dark atrophic acne scars on his forehead for many years, started a (8.8% BE/12% CPO) cream formulation treatment prepared by the method of Example 1, applying it twice daily. After 4 days, the dark scar had reduced visibly (FIG. 12A).

### Example 30

An approximately 25 year old female, with atrophic acne scars on her face for many years, started a (8.8% BE/12% CPO) cream formulation treatment prepared by the method of Example 1, applying it twice daily. After 8 days, the pitted scaring characteristic of acne had visibly reduced with the skin appearing near normal (FIG. 12B).

### Example 31

An approximately 26 year old male, with an open cutaneous injury caused by a hot oil burn on his hand, started a (8.8% BE/12% CPO) cream formulation treatment prepared by the method of Example 1, applying it twice daily. After approximately less than 30 days, the dermal layer had healed with little scaring or hyperkeratosis and near normal appearance (FIG. 12C).

### Example 32

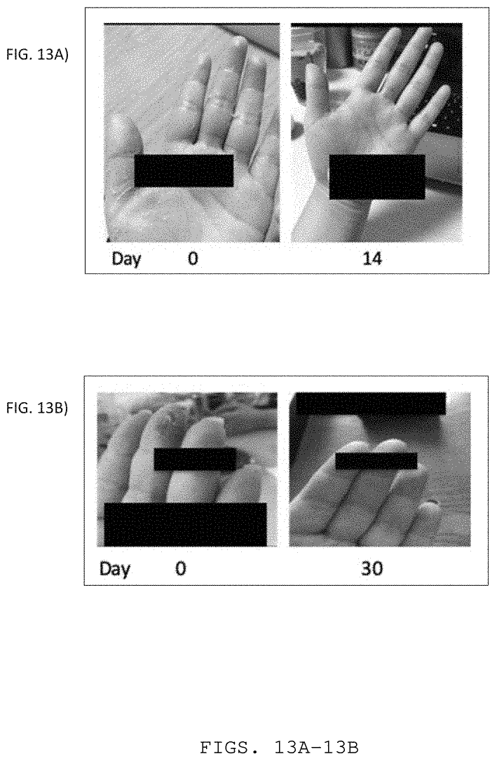

An approximately 30 year old female, with a skin rash on her hands, causing skin peeling, started a (8.8% BE/12% CPO) cream formulation treatment prepared by the method of Example 1, applying it daily. After 14 days, the peeling skin was reduced to normal skin (FIG. 13A).

### Example 33

An approximately 30 year old female, with a skin rash causing her skin to crack and peel on her finger pads, started a (8.8% BE/12% CPO) cream formulation treatment prepared by the method of Example 1, applying it daily. After 30 days, the cracked and peeled finger pad had healed (FIG. 13B).

### Example 34

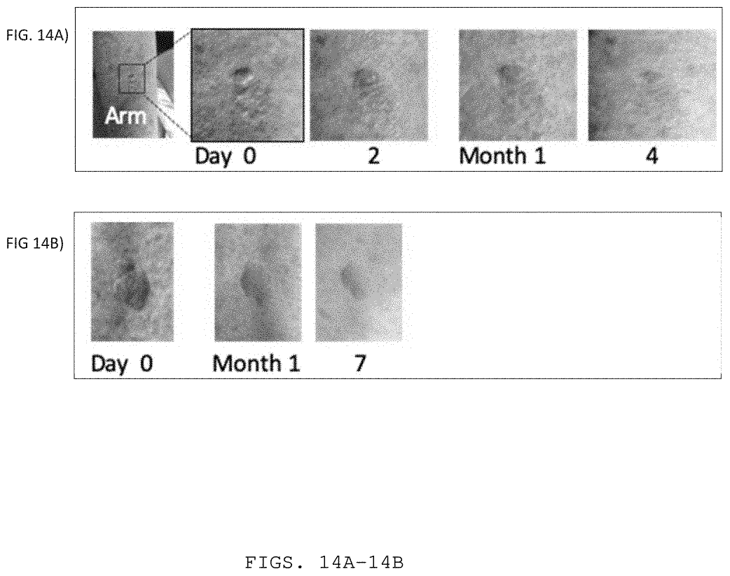

An approximately 79 year old female, with diagnosed seborrheic dermatitis on her arm with characteristic seborrheic keratoses, slightly raised and thickened, wart-like surface, started a (8% BE/12% CPO) cream formulation treatment prepared by the method of Example 1, applying it daily. After 1 month, the raised and thickened lesion had flattened and further still at 4 months (FIG. 14A).

### Example 35

An approximately 69 year old female, with diagnosed seborrheic dermatitis on her leg with characteristic seborrheic keratoses, slightly raised and thickened, wart-like surface, started a (8% BE/12% CPO) cream formulation treatment prepared by the method of Example 1, applying it twice daily. After 1 month, the raised and thickened lesion had flattened and further still at 7 months (FIG. 14B).

What is claimed is:

1. A composition comprising one or more botanical extracts and one or more cold-pressed oils, wherein:
(a) the one or more botanical extracts is selected from the group consisting of pomegranate fruit (Punica granatum) extract, oats (Avena sativa) extract, pennywort (Centella asiatica) extract, sun flower seed (Helianthus annuus) extract, lemon balm (Melissa officinalis) extract, and star anise (Illicium verum) extract; and
(b) the one or more cold-pressed oils is selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

2. The composition of claim 2, wherein the one or more botanical extracts are selected from the group consisting of aqueous botanical extracts, fermented botanical extracts, aqueous/alcoholic botanical extracts, alcoholic botanical extracts, organic solvent botanical extracts, enzymatic botanical extracts, acidic botanical extracts, pressure extracted botanical extracts, botanical extracts prepared by distillation, and botanical extracts prepared via saponification.

3. The composition of claim 3, wherein the one or more botanical extracts are aqueous botanical extracts and fermented botanical extracts.

4. The composition of claims 1-3, wherein the composition is in the form of an emulsion.

5. The composition of claims 1-4, wherein the composition comprises at least two, at least three, at least four, at least five, or all six botanical extracts.

6. The composition of claims 1-5, wherein the emulsion comprises at least two, at least three, at least four, at least five, at least six, at least seven, at least eight, or all nine cold-pressed oils.

7. The composition of claims 2-6, wherein the fermented botanical extracts have been fermented with lactic acid bacteria.

8. The composition of claim 7, wherein the lactic acid bacteria is selected from the group consisting of Lactobacillus sakei, Lactobacillus brevis and combinations thereof.

9. The composition of claims 2-8, wherein the aqueous botanical extracts are prepared by heating one or more of pomegranate fruit, oats, pennywort, sun flower seed, lemon balm, or star anise botanical material in aqueous solution at 100° C. for about 45 minutes or more.

10. The composition of claims 1-9, wherein the composition comprises about 2.5-8.8% by volume of botanical extract and about 12-14% by volume of cold-pressed oil.

11. The composition of claims 1-10, further comprising pro-vitamin B5, tea tree (Melaleuca alternifolia) oil, jojoba (Simmondsia chinensis) oil, and/or argan (Argania spinose) oil.

12. The composition of claims 1-11, comprising a 1:1 ratio of aqueous botanical extract and fermented botanical extract.

13. The composition of claims 1-12, wherein the composition is formulated as a paste, gel, cream, lotion, powder, soap, mask, or spray.

14. The composition of claims 1-13, wherein the composition is formulated as a skin or hair care product.

15. The composition of claims 1-14, wherein the composition is formulated for topical administration.

16. The composition of claims 1-15, wherein the composition is formulated as a lotion or cream.

17. A method of manufacturing the composition of claims 1-16, the method comprising the following steps:
(a) obtaining one or more aqueous botanical extracts by heating an aqueous solution comprising botanical material from one or more plants selected from pomegranate fruit (Punica granatum), oats (Avena sativa), pennywort (Centella asiatica), sun flower seed (Helianthus annuus), lemon balm (Melissa officinalis), and star anise (Illicium verum);
(b) obtaining one or more fermented botanical extracts by fermenting an aqueous solution comprising botanical material from one or more plants selected from pomegranate fruit, oats, pennywort, sun flower seed, lemon balm, and star anise;
(c) removing insoluble material from the one or more botanical extracts obtained in steps (a) and (b) and combining the resultant extracts, or combining the botanical extracts of (a) and (b) and removing insoluble material from the combined botanical extract; and
(d) emulsifying the product of step (c) with one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

18. The method of claim 17, wherein step (c) further comprises emulsifying the botanical extract after removal of the insoluble material with one or more cosmetically acceptable solid oils, humectants, thickeners, and emulsifying agents.

19. The method of claims 17-18, wherein the fermented botanical extracts of step (b) is fermented with lactic acid bacteria.

20. The method of claim 19, wherein the fermentation is performed at about 20-24° C. for about 36 to 72 hours.

21. The method of claims 17-20, wherein the fermentation of one or more botanical extracts is stopped by heat inactivation.

22. The method of claims 17-21, wherein the heating of step (a) is at about 100° C. for about 45 minutes or longer.

23. The method of claims 17-22, wherein the insoluble material in step (c) is removed by filtration or centrifugation.

24. The method of claims 17-23, wherein provitamin B5, tea tree (Melaleuca alternifolia) oil, jojoba (Simmondsia chinensis) oil, argan (Argania spinose) oil, and/or vitamin E oil is added to the one or more aqueous botanical extracts prior to step (c).

25. The method of claims 17-24, wherein the one or more aqueous botanical extracts are cooled to 40° C. or less prior to step (d).

26. A method of manufacturing the composition of claims 1-16, comprising the following steps:
(a) providing one or more botanical extracts;
(b) optionally removing insoluble material from the one or more botanical extracts obtained in step (a); and
(c) emulsifying the product of step (b) with one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

27. The method of claim 26, wherein the one or more botanicals extract of step (a) have been prepared by aqueous extraction, fermentation, aqueous/alcoholic extraction, alcoholic extraction, organic solvent extraction, enzymatic extraction, acidic (e.g., vinegar) extraction, pressure extraction, extraction via distillation, and/or extraction via saponification.

28. A method of treating a subject having a skin disease, disorder or condition comprising topically administering the composition of claims 1-16 to the subject.

29. The method of claim 28, wherein the composition is administered to the subject one or more times per day.

30. The method of claims 28-29, wherein the composition is a cream or lotion.

31. The method of claims 28-30, wherein the skin disease, disorder or condition is selected from the group consisting of hidradenitis suppurativa (HS) acne inversa, psoriasis, eczema, dry skin, sunburn, soft tissue injury, insect bite, radiation dermatitis, birth marks and moles, planter's wart, skin rash, acne vulgaris, dermal scaring and keloids from acne, dermal scaring and keloids from cutaneous burn injuries, skin peeling and cracking, and seborrheic dermatitis.

32. A topical composition comprising extracts of pomegranate fruit (Punica granatum), oats (Avena sativa), pennywort (Centella asiatica), sun flower seed (Helianthus annuus), lemon balm (Melissa officinalis), and star anise (Illicium verum).

33. The composition of claim 31, wherein the composition is prepared by a method comprising:
(a) adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water, and heating the water to 100□C for 45 minutes to prepare an aqueous extract;
(b) adding, by weight, 0.001% to 10% of pomegranate fruit, 0.001% to 20% oats, 0.001% to 5% of pennywort, 0.001% to 2% of sunflower seeds, 0.001% to 1% of lemon balm, 0.001% to 1% of star anise to water to prepare a mixture, adding Lactobacillus sakei to the mixture, and fermenting the mixture at 20□C to prepare a fermented extract; and
(c) combining the aqueous extract and fermented extract at a 1:1 ratio.

34. The composition of claims 32-33, further comprising one or more cold-pressed oils selected from the group consisting of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

35. The composition of claim 34, wherein the composition comprises about 12% cold-pressed oils, wherein the cold-pressed oils comprise a mixture of aloe vera (barbados aloe) oil, rosehip (Rosa canina) oil, sea buckthorn seed (Hippophae rhamnoides) oil, pumpkin (Cucurbita pepo) oil, pomegranate seed (Punica granatum) oil, carrot seed (Daucus carota) oil, chamomile (Matricaria chamomilla) oil, coffee bean (Coffea arabica) oil, raspberry seed (Rubus idaeus) oil, and Vitamin E oil (gamma-tocopherol).

36. The composition of claims 32-35, wherein the composition is formulated as a paste, gel, cream, lotion, powder, soap, mask, bath salt or spray.

37. The composition of claims 32-36, wherein the composition is formulated as a skin or hair care product.

38. The composition of claims 32-37, wherein the composition is formulated for topical administration.

39. The composition of claims 32-38, wherein the composition is formulated as a lotion or cream.

40. The composition of claims 32-33, further comprising magnesium sulfate.
